(19) United States
(12) Patent Application Publication
(10) Pub. No.: US 2022/0088255 A1
(43) Pub. Date: Mar. 24, 2022

(54) SYSTEMS AND METHODS FOR SANITIZING AN INDOOR ENVIRONMENT

(71) Applicant: Aerus, LLC, Dallas, TX (US)

(72) Inventors: Timothy Raymond Vadnais, Plano, TX (US); Andrew Eide, Rockwall, TX (US)

(21) Appl. No.: 17/481,081

(22) Filed: Sep. 21, 2021

Related U.S. Application Data
(60) Provisional application No. 63/081,576, filed on Sep. 22, 2020.

(51) Int. Cl. A61L 9/04; A61L 2/22; A61L 2/20; A61L 2/26; B08B 5/04; B08B 7/00; C11D 11/00

(52) U.S. Cl. CPC A61L 9/044; A61L 2/22; A61L 2/20; A61L 2/26; B08B 5/04; A61L 2202/16; C11D 11/0017; A61L 2202/25; A61L 2209/21; A61L 2202/11; B08B 7/0014

(57) ABSTRACT

A method for sanitizing an indoor environment includes placing a high-output hydroxyl generator within an indoor environment and commencing a hydroxyl shock treatment of the indoor environment by activating the high-output hydroxyl generator. The method also includes applying a disinfectant to hard surfaces or soft surfaces within the indoor environment and applying a microbial growth prevention agent to flooring or the hard surfaces within the indoor environment. The method also includes deactivating the high-output hydroxyl generator and removing the high-output hydroxyl generator from the indoor environment.

### CROSS-REFERENCE TO RELATED APPLICATIONS

This application claims priority to and the benefit of U.S. Provisional Patent Application No. 63/081,576, filed Sep. 22, 2020, the entirety of which is incorporated herein by this reference.

### BACKGROUND

Indoor environments (e.g., rooms within buildings) enable myriad uses, such as residential, commercial, healthcare, athletic, storage, assembly, and/or other uses. However, many microbes (e.g., bacteria, archaea, fungi, protozoa, algae, viruses, etc.), other pathogens, and/or allergens are able to reside and/or grow within indoor environments. In some instances, harmful microbes, pathogens, and/or allergens may continue to reside/grow within interior spaces despite diligent cleaning efforts. For instance, an interior space may include cracks, crevices, and/or other difficult-to-reach areas within which harmful microbes, pathogens, and/or allergens may reside/grow.

Furthermore, in some instances, harmful microbes, pathogens, and/or allergens may rapidly repopulate a previously cleaned surface or area within an indoor environment, particularly where the surface/area is highly trafficked (e.g., regularly handled surfaces, such as door handles, exercise equipment, light switches, etc.).

Accordingly, there are a number of difficulties associated with maintaining clean indoor environments that can be addressed.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

The present disclosure is directed to systems and methods for sanitizing an indoor environment. For example, in at least one embodiment, a method for sanitizing an indoor environment includes placing a high-output hydroxyl generator within an indoor environment and commencing a hydroxyl shock treatment of the indoor environment by activating the high-output hydroxyl generator. The method also includes applying a disinfectant to hard surfaces and/or soft surfaces within the indoor environment and applying a microbial growth prevention agent to flooring and/or other hard surfaces within the indoor environment. The method also includes deactivating the high-output hydroxyl generator and removing the high-output hydroxyl generator from the indoor environment.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be apparent to one of ordinary skill in the art from the description or may be learned by the practice of the teachings herein. Features and advantages of embodiments described herein may be realized and obtained by means of the instruments and combinations particularly pointed out in the appended claims. Features of the embodiments described herein will become more fully apparent from the following description and appended claims.

### DETAILED DESCRIPTION

The present disclosure is directed to systems and methods for sanitizing an indoor environment.

For example, in at least one embodiment, a method for sanitizing an indoor environment includes placing a high-output hydroxyl generator within an indoor environment and commencing a hydroxyl shock treatment of the indoor environment by activating the high-output hydroxyl generator. The method also includes applying a disinfectant to hard surfaces and/or soft surfaces within the indoor environment and applying a microbial growth prevention agent to flooring and/or other hard surfaces within the indoor environment. The method also includes deactivating the high-output hydroxyl generator and removing the high-output hydroxyl generator from the indoor environment.

The embodiments disclosed and claimed herein can provide systems and methods for sanitizing an indoor environment in an advantageous manner. For instance, utilizing a high-output hydroxyl generator to perform a hydroxyl shock treatment within an indoor environment may significantly reduce or eliminate microbes (e.g., fungi, bacteria, viruses), and/or other types of contaminants residing within cracks/crevices of the indoor environment. Furthermore, a hydroxyl shock treatment may be configured to be environmentally friendly and safe for humans, pets, and/or plants. Accordingly, while performing the hydroxyl shock treatment, users may perform other sanitization steps disclosed herein in an efficient manner in parallel with the hydroxyl shock treatment and without having to wait for the hydroxyl shock treatment to end.

For example, as described herein, users may vacuum surfaces, apply disinfectant to the surfaces, apply carpet shampoo to carpets (e.g., while the disinfectant dries), apply a microbial growth prevention agent to flooring/surfaces (e.g., while the carpet shampoo dries), vacuum the carpets, apply a microbial growth prevention agent to the carpet, and/or place a long-term air purification system within the indoor environment while the hydroxyl shock treatment occurs. Allowing the hydroxyl shock treatment to occur while performing other sanitization acts may provide an efficient process for sanitizing an indoor environment, where multiple sanitization steps can occur simultaneously. Furthermore, in some instances, the sanitization acts described herein may be performed in an optimal order to promote sanitization efficiency (e.g., to prevent delays that may occur while waiting for treated surfaces to dry).

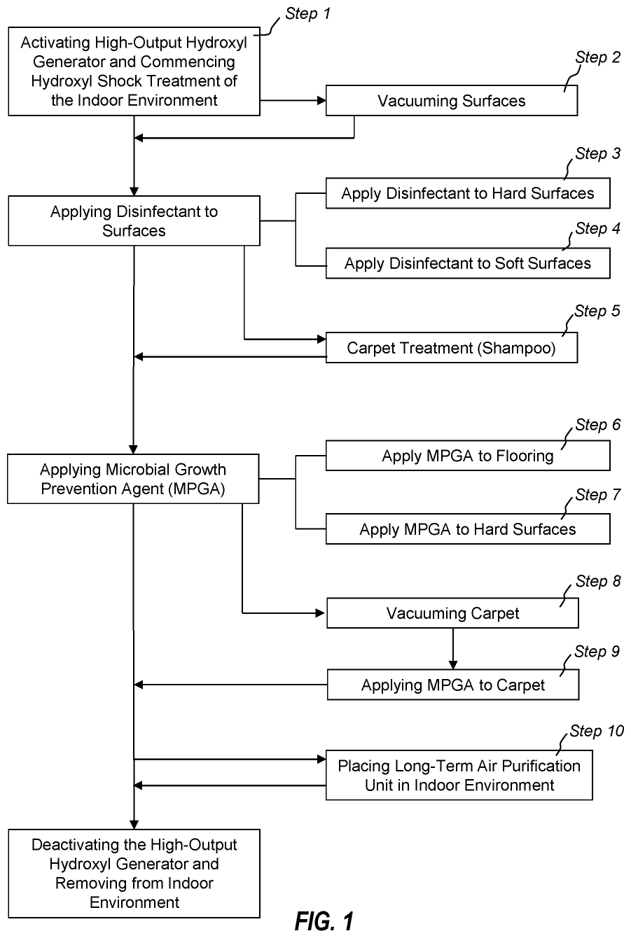

With reference to FIG. 1, the following discussion now refers to a number of acts associated with sanitizing an indoor environment (e.g., method acts). Although the acts may be discussed in a certain order as part of one exemplary, preferred embodiment, or given ordinal associations for ease of description (e.g., step 1, step 2, etc.), no particular ordering is required unless specifically stated, or required because an act is dependent on another act being completed prior to the act being performed. Furthermore, it should be noted that, in some instances, at least some of the acts described hereinbelow for sanitizing an indoor environment may be omitted or replaced with alternative acts, in accordance with implementations of the present disclosure. In addition, in some instances, additional acts not explicitly described herein may be perform in combination with the acts described hereinbelow for sanitizing an indoor environment.

In some instances, step 1 for sanitizing an indoor environment includes commencing a hydroxyl shock treatment within the indoor environment. Step 1 may include, for example, placing a high-output hydroxyl generator within the indoor environment, connecting the high-output hydroxyl generator to a power source (e.g., an electrical outlet within the indoor environment), and activating or otherwise turning on the high-output hydroxyl generator to commence the hydroxyl shock treatment.

A high-output hydroxyl generator may take on any suitable form in accordance with implementations of the present disclosure, such as an ActivePure hydroxyl blaster, provided by Aerus LLC and/or associated entities. In some instances, the high-output hydroxyl generator generates hydroxyls through photocatalytic oxidation (PCO) and/or an advanced oxidation process (AOP) (also referred to as direct hydroxyl production (DHP)) at a sufficient rate and in combination with sufficient airflow to effectively disperse hydroxyls and sanitize a desired treatment area within the indoor environment. For example, a hydroxyl shock treatment (often effectuated with a dedicated type of high-output hydroxyl generator) may be distinguished from passive or continuous hydroxyl release for continuous, long-term air treatment of an indoor environment by a permanent or semi-permanent low-output hydroxyl release device (e.g., such as hydroxyls potentially released by a long-term air purification system, as described hereinbelow with reference to step 10). A hydroxyl shock treatment may involve the generation of significantly greater amounts of hydroxyls as compared to a permanent or semi-permanent low-output hydroxyl release device, such as a hydroxyl generation rate that is about 5× or more, 10× or more, or 20× or more than with a permanent or semi-permanent low-output hydroxyl release device. In some instances, a high-output hydroxyl generator is configured to sanitize an indoor environment or portion thereof (e.g., a room within a range of up to about 3,000 sq. ft. to about 7,000 sq. ft. with a ceiling height within a range of about 8 ft. to about 10 ft. or higher) within a time period of 12 hours or less (e.g., 10 hours, 8 hours, 6 hours, 4 hours, 2 hours, 1 hour, or a different time period).

In at least some instances, a hydroxyl shock treatment is configured to significantly reduce or eliminate microbes and/or other types of contaminants that are airborne and/or positioned in hard-to-reach places within the indoor environment (e.g., within cracks, corners, crevices, etc.). Initiating the hydroxyl shock treatment as a first step in the sanitization process may maximize the sanitizing effects of the hydroxyl shock treatment within the indoor environment (e.g., allowing the hydroxyl shock treatment to occur for about one hour, longer than about one hour, longer than about two hours, longer than about 3 hours, or for any time period that constitutes the duration of the sanitization process for the indoor environment).

Furthermore, although hydroxyls are effective in reducing or eliminating microbes and contaminants, a hydroxyl shock treatment is typically environmentally friendly and safe to perform in the presence of humans, animals, and/or plants. In this regard, after commencing the hydroxyl shock treatment in accordance with step 1, one or more users may perform other sanitization activities within the indoor environment while the hydroxyl shock treatment occurs, allowing the overall process to be performed in an efficient manner.

In some instances, step 2 for sanitizing an indoor environment includes vacuuming surfaces within the indoor environment. As indicated above, one or more users may perform step 2 while the hydroxyl shock treatment associated with step 1 is occurring. Step 2 may involve vacuuming hard surfaces within the indoor environment, soft surfaces within the indoor environment, and/or flooring within the indoor environment (e.g., carpet flooring and/or non-carpet flooring).

In some instances, a user utilizes a vacuum cleaner that includes one or more filters that comply with high efficiency particulate air (HEPA) filter standards. In some instances, to improve efficiency, the vacuum cleaner may comprise a canister-style vacuum and/or may include or be connected to a vacuum hose that is long enough (e.g., 12 feet) to allow the user to vacuum various hard surfaces and/or soft surfaces within the indoor environment without requiring the user to perform excessive intermittent movements of the main body of the vacuum cleaner. In other instances, the vacuum cleaner is at least partially wearable by users (e.g., a backpack-style vacuum cleaner), allowing the user fluid maneuverability while vacuuming to reach hard surfaces and/or soft surfaces within the indoor environment.

As used herein, “hard surfaces” may comprise any number of walls, tables, desks, counter tops, chairs, dressers, other wood-based/polymer/metallic furnishings or fixtures, light switches, remote controls, telephones, actuatable elements (e.g., buttons, levers, window/door locks), thermostats, door/window and/or other appliance knobs or handles, equipment (e.g., exercise equipment, such as hand weights, treadmills, weights, bars, free weights, etc.), machinery, and/or other rigid surfaces within the indoor environment.

As used herein, “soft surfaces” may comprise any number of upholstered portions of furnishings, mattresses, pillows, window/partition coverings (e.g., drapes/curtains), fabric layers (e.g., fabric layers on walls), and/or other flexible materials/surfaces within the indoor environment.

In some instances, vacuuming the hard surfaces and/or soft surfaces within the indoor environment according to step 2 removes dirt and/or debris from the surfaces to prepare the surfaces for disinfection and/or other treatments described herein.

For example, in some instances, step 3 for sanitizing an indoor environment includes applying a disinfectant to the hard surfaces within the indoor environment. As with step 2, a user may also perform step 3 while the hydroxyl shock treatment of the indoor environment is occurring according to step 1. Various disinfectants for eliminating contaminants (e.g., microbes, pathogens, allergens) are within the scope of this disclosure for performing step 3, such as, for example, ActiveClean solution, provided by Aerus LLC and/or associated entities.

A user may perform step 3 by spraying and/or wiping the disinfectant onto the hard surfaces within the indoor environment. In some implementations (e.g., to provide deep cleaning), a user applies the disinfectant to the hard surfaces within the indoor environment by first spraying the disinfectant onto the hard surfaces and subsequently wiping the hard surfaces with an applicator (e.g., a microfiber cloth) that is saturated with the disinfectant. For example, a user may utilize a spray bottle (or other apparatus, such as a backpack sprayer or other form of spray device) to spray the disinfectant onto the hard surface (e.g., from a distance of 6 to 8 inches). The user may then refrain from further treating the hard surface for a period of time (e.g., 5 minutes) and may optionally perform other sanitization operations while waiting (e.g., by treating additional hard surfaces according to step 3, and/or by initiating portions of other steps described herein). After the period of time has passed, the user may utilize an applicator (e.g., a microfiber cloth) that is saturated with the disinfectant to again treat the hard surface by wiping the hard surface with the disinfectant-saturated applicator.

In other implementations, such as where the hard surface to be treated is only lightly trafficked or is not especially dirty, a user may either spray the hard surface with disinfectant without performing the additional steps of waiting for a time period and wiping (with a disinfectant-saturated applicator), or the user may wipe the hard surface with a disinfectant-saturated applicator and refrain from performing the initial steps of spraying the hard surface with the disinfectant and waiting for a time period.

In some instances, hard surfaces treated according to step 3 become dry within about five minutes to about ten minutes of application of the disinfectant. Accordingly, immediately after treatment according to step 3, a user may proceed with other steps described herein in an efficient manner.

In some instances, step 4 for sanitizing an indoor environment includes applying the disinfectant to the soft surfaces within the indoor environment. As with steps 2 and 3, a user may also perform step 4 while the hydroxyl shock treatment of the indoor environment is occurring according to step 1. Similar to step 3 described hereinabove, various disinfectants effective for reducing or eliminating contaminants (e.g., microbes, pathogens, allergens), without damaging the surfaces to which they are applied, are within the scope of this disclosure for performing step 3. An example of an effective disinfectant is ActiveClean solution, provided by Aerus LLC and/or associated entities.

In some instances, steps 3 and 4 are performed using the same disinfectant, whereas in other implementations, at least some surfaces treated according to steps 3 and 4 are treated using different disinfectants. Furthermore, it should be noted that, in some implementations, a user may utilize different disinfectants for treating different hard surfaces (according to step 3) and/or may utilize different disinfectants for treating different soft surfaces (according to step 4). However, it should also be noted that, in some instances, utilizing the same disinfectant for treating surfaces according to steps 3 and/or 4 may provide efficiency gains by avoiding intermittent changes in applicator/disinfectant while sanitizing the indoor environment.

In some instances, a user applies the disinfectant to the soft surfaces within the indoor environment according to step 4 by dispersing a coat of disinfectant onto the soft surfaces using a spraying device (e.g., a spray bottle, backpack sprayer, and/or other spraying device) or a fogging device (e.g., an ultra-low volume (ULV) fogging device). For example, a user may disperse the coat of disinfectant onto the soft surfaces using the spraying or fogging device from a distance of about 6 to 8 inches from the soft surface being coated. Advantageously, in some instances, a user may omit a step of wiping the soft surfaces in accordance with step 4 as described herein, allowing expeditious disinfectant treatment of the soft surfaces within the indoor environment.

Furthermore, similar to step 3, the soft surfaces treated in accordance with step 4 often dry within about five minutes to about ten minutes, allowing users to proceed with other steps described herein immediately after treatment in an efficient manner. It should be noted that at least some methods for sanitizing an indoor environment according to the present disclosure include additional treatments to the hard surfaces and the soft surfaces within the indoor environment (see, e.g., steps 6 and 7 described hereinbelow). However, in some instances, the results of the additional treatments to the hard surfaces and the soft surfaces within the indoor environment are improved by first allowing the hard surfaces and the soft surfaces to dry after being treated in accordance with steps 3 and 4 described above. Accordingly, in some implementations, performing the acts for sanitizing an indoor environment in the order prescribed (e.g., step 1, step 2, step 3, etc.) may allow users to be continuously engaged in sanitizing activities in an efficient manner, rather than idly waiting for treated surfaces to dry before performing subsequent treatments to the same surfaces.

In some instances, step 5 includes initiating a carpet treatment process to treat carpet(s) (if any) within the indoor environment. As with steps 2, 3, and 4, a user may also perform step 5 while the hydroxyl shock treatment of the indoor environment is occurring. Furthermore, as indicated above, a user may commence step 5 after disinfecting hard surfaces (according to step 3) and/or soft surfaces (according to step 4) within the environment and while the treated surfaces are drying. In this regard, a user may use time efficiently while allowing the surfaces treated according to steps 3 and/or 4 to dry in preparation for applying a microbial growth prevention agent to the surfaces (e.g., according to step 7) in an efficient manner.

The carpet treatment process of step 5 may include applying a carpet shampoo to carpets within the indoor environment. Various carpet shampoos operable to clean and/or shampoo a carpet are within the scope of this disclosure. For example, a carpet shampoo for performing step 5 may comprise a crystal drying carpet shampoo that captures/traps dust mites, allergens, and/or odor causing particles within crystals that form upon drying, such as Turbo Shampoo, provided by Aerus LLC and/or associated entities. Similarly, a user may employ various carpet shampoo application devices in accordance with step 5, such as, for example, a Lux Floor Pro shampooer device, provided by Aerus LLC and/or associated entities.

A user may follow prescribed ratio and mixing guidelines for the carpet shampoo and add the shampoo solution to a reservoir or tank of a carpet shampooing device. In some instances, a user begins shampooing the carpet in a corner of the carpet that is remote from an entrance or door of the room in which the carpet resides and works outward from the corner. A user may apply the carpet shampoo under a sectioned approach (e.g., working sequentially in 4-foot by 4-foot sections). After applying the carpet shampoo to the carpet, a user may efficiently proceed with other sanitization steps while the carpet dries (which may take up to one hour or longer) and before performing additional carpet treatments (e.g., additional treatments that are optimally performed on dry carpet, such as step 9 described hereinbelow).

For example, while allowing the carpet shampoo to dry, a user may proceed to step 6 of sanitizing an indoor environment, which, in some instances, includes applying a microbial growth prevention agent (or other protectant or long-term disinfectant) to non-carpet flooring (if any) within the indoor environment. As with steps 2, 3, 4, and 5, a user may also perform step 6 while the hydroxyl shock treatment of the indoor environment is occurring.

Various microbial growth prevention agents (or other protectants or long-term disinfectants) for preventing/inhibiting the growth of microorganisms, pathogens, and/or allergens associated with such microbes are within the scope of this disclosure, such as, for example, ActiveShield or AllerGuard, provided by Aerus LLC and/or associated entities. In some instances, a microbial growth prevention agent may inhibit/impede growth of contaminants for a substantial period of time (e.g., up to three months or six months).

As used herein, “non-carpet flooring” within the indoor environment may refer, for example, to rubberized flooring, tile flooring, stone flooring, laminate flooring, vinyl flooring, linoleum flooring, hardwood flooring, concrete flooring, and/or others.

In some instances, a user applies the microbial growth prevention agent to the non-carpet flooring within the indoor environment, according to step 6, by dispersing a coat of the microbial growth prevention agent onto the non-carpet flooring using a spraying device (e.g., a spray bottle, backpack spray, and/or other spraying device) or a fogging device (e.g., a ULV fogging device). For example, a user may spray/disperse the microbial growth prevention agent directly onto the non-carpet flooring from a height of about 12 inches to 24 inches above the non-carpet flooring. Furthermore, in some instances, a user may utilize a spreading device (e.g., a microfiber mop) to evenly spread the microbial growth prevention over the non-carpet flooring, creating an even coat of the microbial growth prevention agent on the non-carpet flooring.

After treatment, a user may efficiently proceed with other sanitization steps described herein while the microbial growth prevention agent dries on the non-carpet flooring (which may take, for example, about five minutes to about ten minutes).

In some instances, step 7 for sanitizing an indoor environment includes applying a microbial growth prevention agent to the hard surfaces within the indoor environment. As with steps 2, 3, 4, 5, and 6, a user may also perform step 7 while the hydroxyl shock treatment of the indoor environment is occurring according to step 1. Similar to step 6 described hereinabove, various microbial growth prevention agents (or other protectants or long-term disinfectants) are within the scope of this disclosure, such as, for example, ActiveShield or AllerGuard, provided by Aerus LLC and/or associated entities.

In some instances, steps 6 and 7 are performed using the same microbial growth prevention agent, whereas in other implementations, at least some flooring and/or hard surfaces treated according to steps 6 and 7 are treated using different microbial growth prevention agents. Furthermore, it should be noted that, in some implementations, a user may utilize different microbial growth prevention agents for treating different types of non-carpet flooring (according to step 6) and/or may utilize different microbial growth prevention agents for treating different hard surfaces (according to step 7). However, it should also be noted that, in some instances, utilizing the same microbial growth prevention agent for treating the non-carpet flooring and the hard surfaces within the indoor environment may provide efficiency gains by avoiding intermittent changes in applicator and/or microbial growth prevention agent while sanitizing the indoor environment.

In some implementations, a user applies the microbial growth prevention agent to the hard surfaces within the indoor environment by spraying (or otherwise applying) the microbial growth prevention agent onto an applicator (e.g., a microfiber cloth) and wiping the hard surfaces with the applicator. In some instances, a user may refrain from utilizing applicators that include abrasive elements, which may reduce and/or render uneven the coverage of the microbial growth prevention agent when applied to the hard surfaces.

In some instances, applying the microbial growth prevention agent to the hard surfaces by wiping, rather than spraying, can prevent excessive or unintended application of the microbial growth prevention agent, while still providing an even spread of the microbial growth prevention agent over the hard surfaces. For example, at least some microbial growth prevention agents may cause undesirable effects when applied to some surfaces, such as fogging/clouding when applied to transmissive or reflective surfaces (e.g., glass, chrome, etc.); such undesirable effects may at least partially be avoided by refraining from spraying the microbial growth prevention agent directly onto the hard surfaces within the indoor environment.

After treatment, a user may efficiently proceed with other sanitization steps described herein while the microbial growth prevention agent dries on the hard surfaces (which may take, for example, about five minutes to about ten minutes).

In some instances, step 8 for sanitizing an indoor environment includes vacuuming the carpet(s) within the indoor environment. As with steps 2, 3, 4, 5, 6, and 7, a user may also perform step 8 while the hydroxyl shock treatment of the indoor environment is occurring according to step 1. In some instances, step 8 may be regarded as continuing the carpet treatment process that was initiated by applying the carpet shampoo to the carpet as part of step 5 described hereinabove. In this regard, a user may commence step 8 after sufficient time has been allowed for the carpet(s) to dry after having the carpet shampoo applied thereto. For example, in implementations that include a crystal drying shampoo, a user may commence step 8 after crystals have formed to capture/trap dust mites, allergens, and/or odor causing particles from the carpet. Advantageously, as described hereinabove, a user may have performed other steps for sanitizing the indoor environment (e.g., steps 6 and 7) to efficiently occupy the drying time for the carpet before performing step 8.

Similar to step 2 described hereinabove, a user may perform step 8 utilizing a vacuum cleaner that includes one or more HEPA filters. In some instances, a user performs step 8 utilizing the same vacuum cleaner(s) used to perform step 2 described hereinabove, whereas in other instances, different vacuum cleaners are used. As indicated above, in implementations that include a crystal drying shampoo, vacuuming the carpet in accordance with step 8 may include vacuuming the crystals formed by the crystal drying shampoo, which may remove dirt, debris, allergens, dust mites, odor causing particles, and/or other contaminants trapped within the crystals or otherwise present in the carpet. Vacuuming the carpet in accordance with step 8 may prepare the carpet for additional treatment (e.g., microbial growth prevention treatment, see step 9).

In some instances, step 9 for sanitizing an indoor environment includes applying a microbial growth prevention agent to the carpet within the indoor environment. As indicated above, step 9 may be performed after preparing the carpet(s) by vacuuming according to step 8 described above. Furthermore, as with steps 2, 3, 4, 5, 6, 7, and 8, a user may also perform step 9 while the hydroxyl shock treatment of the indoor environment is occurring according to step 1. Step 9 may also be regarded as continuing the carpet treatment process that was initiated by applying the carpet shampoo to the carpet as part of step 5 described hereinabove. In some instances, a user performs step 9 using the same microbial growth prevention agent described hereinabove with reference to steps 6 and 7, however a user may also utilize any number of different microbial growth prevention agents for step 9, in accordance with the present disclosure.

In some instances, a user applies the microbial growth prevention agent to the carpet(s) within the indoor environment using a spraying device (e.g., a spray bottle, backpack spray, and/or other spraying device) or a fogging device (e.g., a ULV fogging device). For example, a user may spray/disperse the microbial growth prevention agent directly onto the carpet from a height of about 12 inches to 24 inches above the carpet. After treatment, a user may efficiently proceed with other sanitization steps described herein while the microbial growth prevention agent dries on the carpet(s) (which may take, for example, about five minutes to about ten minutes).

In some instances, step 10 for sanitizing an indoor environment includes placing a long-term air purification system within the indoor environment. As with steps 2, 3, 4, 5, 6, 7, 8, and 9, a user may also perform step 10 while the hydroxyl shock treatment of the indoor environment is occurring according to step 1. Various long-term air purification systems for eliminating airborne (or settled) contaminants are within the scope of this disclosure. For example, a user may place and activate an air purification system within the indoor environment that uses air filtration (e.g., using a HEPA filter or better-than-HEPA filter), photocatalytic oxidation (e.g., to continue to release hydroxyls to eliminate and/or prevent growth of microbes/contaminants), and/or other techniques to maintain and/or improve air quality within the indoor environment in an ongoing manner. Examples of such devices include the Beyond Guardian Air device and/or the AP500 device, provided by Aerus LLC and/or associated entities.

In some instances, a user performs step 10 by placing the long-term air purification system at a sufficient distance from walls (e.g., about 6 inches or more) to allow sufficient airflow to maintain purified air within the indoor environment (even after the method steps for sanitizing an indoor environment are complete). For example, in some instances, the long-term air purification system is a portable air purification system that allows users to easily relocate/reposition the air purification system within the indoor environment and advantageously allows users to install/place the air purification system without performing permanent installation procedures.

The user may also set the long-term air purification system to a desired setting (e.g., a maximum air change per hour setting) to allow sufficient airflow to maintain purified air within the indoor environment. In some instances, subsequent occupants/users of the indoor environment may adjust the setting of the long-term air purification system.

In some implementations, after performing steps 1-10 as described hereinabove, a user may deactivate the high-output hydroxyl generator and therefore end the hydroxyl shock treatment that was initiated according to step 1 as described hereinabove. The user may subsequently remove the high-output hydroxyl generator from the indoor environment. As described above, carrying out the hydroxyl shock treatment while performing all other steps for sanitizing an indoor environment (e.g., steps 2-10 or some subset thereof) allows for simultaneous and more efficient performance of steps for sanitizing an indoor environment. Furthermore, it should be noted that, in some instances, a user may relocate the high-output hydroxyl generator during the hydroxyl shock treatment (e.g., before deactivating the high-output hydroxyl generator) to focus the hydroxyl shock treatment on different portions of the indoor environment (e.g., while performing or in between the performance of steps 2-10 for sanitizing an indoor environment).

Furthermore, it should be noted that, in some instances, a user deactivates the high-output hydroxyl generator before completing all other steps for sanitizing the indoor environment (e.g., steps 2-10). For example, smaller indoor environments may be deemed to have reached sufficient levels of hydroxyls to the point where continued operation of the high-output hydroxyl generator is not necessary.

Those skilled in the art will appreciate, in view of the present disclosure, that the steps for sanitizing an indoor environment as described herein may be selectively modified depending on the needs and/or configuration of the indoor environment (e.g., different types of indoor environments, such as medical environments, sporting environments, office environments, food service environments, residential environments, etc. may have different sanitization needs). For example, one or more users may at least partially refrain from performing step 2, 3, 4, and/or 7 on indoor environments that omit hard surfaces and/or soft surfaces. In another example, one or more users may at least partially refrain from performing steps 5, 8, and/or 9 on indoor environments that omit carpet(s). As yet another example, one or more users may at least partially refrain from performing step 6 on indoor environments that omit non-carpet flooring. Still furthermore, one or more users may at least partially refrain from performing step 10. For example, some indoor environments may be relatively less trafficked and may therefore benefit less from the presence of a continual air purification system, and/or some indoor environments may not be arranged in a manner that is conducive to use of a continual air purification system.

Furthermore, in some instances, one or more users may selectively modify the steps described herein for sanitizing an indoor environment based on whether the steps are being performed as part of an initial cleaning activity or as part of a maintenance cleaning activity. For example, when performing an initial cleaning activity on an indoor environment, one or more users may perform all or substantially all of the steps for sanitizing an indoor environment as described hereinabove (e.g., steps 1-10). Maintenance cleaning activities may then be scheduled based on the date(s) of the initial cleaning activity. For instance, maintenance cleaning activities may be scheduled to occur every 30 days or every month after the initial cleaning activity for a number of months, days, or years.

In some instances, maintenance cleaning activities may comprise performing fewer than all of the steps for sanitizing an indoor environment described hereinabove. For example, at a first maintenance cleaning conducted at some time period following initial cleaning (e.g., 30 days or 1 month after the initial cleaning activities), a maintenance cleaning activity may comprise performing steps 3, 4, 6, and 7 on fewer or all portions of the indoor environment, and servicing any long-term air purification systems positioned within the indoor environment as needed (e.g., cleaning/changing filters, replacing UV bulbs of the long-term air purification systems).

A second maintenance cleaning may be performed at a subsequent time period (e.g., 60 days or 2 months after the initial cleaning activities). The second maintenance cleaning activity may comprise performing steps 2, 3, 4, 6, and 7 on the same set or a different set of portions of the indoor environment that were serviced at the first maintenance cleaning. For example, where the indoor environment includes a specialized room, such as a weight room, the surfaces within the specialized room may be treated at some extended time period (e.g., 60 days or 2 months rather than 30 days or 1 month), and maintenance may further include servicing any long-term air purification systems positioned within the indoor environment as needed.

A third maintenance cleaning may be performed at a subsequent time period (e.g., 90 days or 3 months after initial cleaning activities). The third maintenance cleaning activity may comprise performing steps 2, 3, 4, 5, 6, 7, 8, and 9 on any portions of the indoor environment (e.g., excluding specialized rooms, such as weight rooms, in some implementations), and servicing any long-term air purification systems positioned within the indoor environment as needed.

A fourth maintenance cleaning (e.g., at 120 days or 4 months after initial cleaning activities), a maintenance cleaning activity may comprise performing steps 3, 4, 6, and 7 on any portions of the indoor environment (e.g., excluding specialized rooms, such as weight rooms, in some implementations), and servicing any long-term air purification systems positioned within the indoor environment as needed.

A fifth maintenance cleaning (e.g., at 150 days or 5 months after initial cleaning activities), a maintenance cleaning activity may comprise performing steps 2, 3, 4, 6, and 7 on the same set or a different set of portions of the indoor environment that were serviced at the first maintenance cleaning, and servicing any long-term air purification systems positioned within the indoor environment as needed.

A sixth maintenance cleaning (e.g., at 180 days or 6 months after initial cleaning activities), a maintenance cleaning activity may comprise performing steps 2, 3, 4, 5, 6, 7, 8, and 9 on any portions of the indoor environment (e.g., excluding specialized rooms, such as weight rooms, in some implementations), and servicing any long-term air purification systems positioned within the indoor environment as needed. Those skilled in the art will appreciate, in view of the present disclosure, that the maintenance schedule discussed above is provided as an example only, and that other maintenance schedules are within the scope of this disclosure.

Although the present disclosure references some elements in singular form (e.g., a user, carpet, applicator, fogging device, high-output hydroxyl generator, long-term air purification system, etc.), it should be noted that singular references to elements herein are intended to also encompass pluralities of the elements referenced. It should also be noted that plural references to elements herein (e.g., hard surfaces, soft surfaces, carpets, users, etc.) are intended to also encompass singular elements.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

We claim:

1. A method for sanitizing an indoor environment, comprising:
placing a high-output hydroxyl generator within an indoor environment;
commencing a hydroxyl shock treatment of the indoor environment by activating the high-output hydroxyl generator;
applying a disinfectant to hard surfaces, soft surfaces, or both within the indoor environment;
applying a microbial growth prevention agent to flooring, the hard surfaces, or both within the indoor environment;
deactivating the high-output hydroxyl generator; and
removing the high-output hydroxyl generator from the indoor environment.

2. The method of claim 1, further comprising:
after commencing the hydroxyl shock treatment, vacuuming hard surfaces and soft surfaces within the indoor environment.

3. The method of claim 1, wherein applying the disinfectant to the hard surfaces within the indoor environment comprises spraying the disinfectant onto the hard surfaces and subsequently wiping the hard surfaces with an applicator that is saturated with the disinfectant.

4. The method of claim 1, wherein applying the disinfectant to the soft surfaces within the indoor environment comprises dispersing a coat of the disinfectant onto the soft surfaces using a spraying device or a fogging device.

5. The method of claim 1, wherein applying the microbial growth prevention agent to the flooring within the indoor environment comprises dispersing a coat of the microbial growth prevention agent onto the flooring using a spraying device or a fogging device.

6. The method of claim 1, wherein applying the microbial growth prevention agent to the hard surfaces within the indoor environment comprises spraying the microbial growth prevention agent onto an applicator and wiping the hard surfaces with the applicator.

7. The method of claim 1, further comprising:
after commencing the hydroxyl shock treatment, performing a carpet treatment process that includes:
applying a shampoo to a carpet within the indoor environment;
allowing the shampoo to dry; and
vacuuming the carpet within the indoor environment.

8. The method of claim 7, wherein:
the shampoo is a crystal drying shampoo,
allowing the shampoo to dry comprises allowing the crystal drying shampoo to form crystals, and
vacuuming the carpet within the indoor environment comprises
vacuuming the crystals formed by the crystal drying shampoo.

9. The method of claim 7, wherein applying the shampoo to the carpet is performed after applying the disinfectant to the hard surfaces or the soft surfaces within the indoor environment.

10. The method of claim 7, wherein allowing the shampoo to dry is performed while applying the microbial growth prevention agent to the flooring or the hard surfaces within the indoor environment.

11. The method of claim 7, wherein vacuuming the carpet within the indoor environment is performed after applying the microbial growth prevention agent to the flooring or the hard surfaces within the indoor environment.

12. The method of claim 7, wherein the carpet treatment process further comprises:
after vacuuming the carpet within the indoor environment, applying the microbial growth prevention agent to the carpet within the indoor environment.

13. The method of claim 1, further comprising:
after commencing the hydroxyl shock treatment, placing a long-term air purification system within the indoor environment.

14. The method of claim 1, further comprising:
after commencing the hydroxyl shock treatment but before deactivating the high-output hydroxyl generator, relocating the high-output hydroxyl generator within the indoor environment.
